(19) United States
(12) Patent Application Publication
(10) Pub. No.: US 2010/0144678 A1
(43) Pub. Date: Jun. 10, 2010

(54) COMPOSITIONS AND METHODS FOR TREATING BONE CANCER

(75) Inventors: Lajos Gera, Denver, CO (US); John M. Stewart, Denver, CO (US); Leland W.K. Chung, Los Angeles, CA (US); Daqing Wu, Atlanta, GA (US)

Correspondence Address: SHERIDAN ROSS PC, 1560 BROADWAY, SUITE 1200, DENVER, CO, 80202, US

(73) Assignee: The Regents of the University of colorado, a body corporate, Denver, CO (US)

(21) Appl. No.: 12/595,296

(22) PCT Filed: Apr. 9, 2008
(86) PCT No.: PCT/US08/59732

§ 371 Date: Jan. 6, 2010

Related U.S. Application Data
(60) Provisional application No. 60/910,822, filed on Apr. 9, 2007.
(60) Provisional application No. 60/955,822, filed on Aug. 14, 2007.

(51) Int. Cl. A61K 31/675; C12Q 1/68; C12N 5/02; A61K 31/662; C07F 9/28; A61P 35/04

(52) U.S. Cl. 514/89; 435/6; 435/375; 514/107; 546/22; 568/15

(57) ABSTRACT

Small molecule bradykinin inhibitor bisphosphonate amide derivatives useful for inhibiting cancer growth and treating cancer residing in and around bone are disclosed. These compounds and pharmaceutical compositions containing these compounds are particularly useful for the treatment of prostate cancer bone metastases.

### GOVERNMENT INTEREST

This invention was made with Government support awarded by the National Institutes of Health (NIH) and the Department of Defense. The Government has certain rights in this invention.

### FIELD OF THE INVENTION

The invention relates to the fields of pharmaceuticals and oncology and provides novel methods of treating bone cancer and particularly human prostate cancer skeletal metastasis with bisphosphonates conjugated with derivatives of bradykinin receptor antagonists.

### BACKGROUND OF THE INVENTION

Prostate cancer is the second leading cause of cancer related deaths in males. Initially, prostate cancer growth and progression is dependent upon androgenic hormones. The importance of androgens in prostate cancer is demonstrated by the fact that at least 75% of prostate cancer with metastatic potential is androgen dependent at the time of diagnosis. This androgen dependence has been exploited by several therapies commonly referred to as “androgen ablation therapy.” However, these therapies have considerable side effects. Moreover, most of these therapies eventually fail as prostate cancer progresses to androgen independence, also referred to as “hormone-refractory prostate cancer.” Most prostate cancer deaths result from emergence of this androgen resistant phenotype of prostate cancer. As prostate cancer progresses to advanced stages and androgen independence, it often metastasizes, frequently establishing bone metastasis. To date no satisfactory treatment options are available for these patients with androgen-resistant prostate cancer bone metastases. Thus, there is a great need for new therapies that can prevent and treat prostate cancer in the hormone-dependent and especially in the hormone-independent stages and thereby improve the outlook for patients with prostate cancer that has metastasized to bone.

Bone degeneration diseases, including Paget's Disease and osteoporosis have proven difficult to treat because the mechanisms involved in the development and progression of these diseases are not well understood. Bisphosphonates are synthetic analogs of pyrophosphates characterized by a phosphorus-carbon-phosphorus backbone that renders them resistant to hydrolysis and are known to be useful in the treatment of these degenerative bone disorders. The chemical properties of the bisphosphonates vary based on different substitutions at the carbon atom of the phosphorus-carbon-phosphorus backbone and the presence and identity of any substitutions at the hydroxyl moieties on the phosphorus atoms. Bisphosphonates bind strongly to hydroxyapatite on the bone surface and act to reduce and inhibit the activity of osteoclasts; cells functioning in the resorption and removal of osseous tissue. The anti-resorptive effect of bisphosphonates is also mediated through effects on osteoblasts; cells that function in the production of bone. Thus, biophosphonates are used clinically to inhibit bone resorption in disease states such as Paget's disease, osteoporosis, metastatic bone diseases, and malignant and nonmalignant hypercalcemia. Bisphosphonates are also used to mediate anti-cancer effects by modifying the bone surface, altering the bone microenvironment, inhibiting specific enzymatic pathways and inducing apoptosis in osteoclast and tumor cells.

Bisphosphonates that are currently used therapeutically include alendronate, clodronate, etidronate, pamidronate, tiludronate, ibandronate, zoledronate, olpadronate, residronate and neridronate. Additionally, bone-scanning agents based on the use of bisphosphonic acid compounds have been used in the past to produce high definition bone scans (see e.g., U.S. Pat. No. 4,810,486 to Kelly et al.). Bisphosphonate derivatives have been used as therapeutic agents for bone diseases such as osteoporosis, rheumatoid arthritis, and osteoarthritis (see e.g., U.S. Pat. No. 5,428,181 to Sugioka et al.). In the past, however, bisphosphonate therapies have frequently been accompanied by severe side effects such as retardation of bone development and somatic growth.

Therefore, a need exists for novel bisphosphonate compounds that act as delivery vehicles to target and deliver therapeutic anti-cancer agents to bone and the surrounding soft tissue, allowing selective treatment of these tissues by co-targeting cancer and its bone microenvironment.

### SUMMARY OF THE INVENTION

The present invention is directed to methods of targeting anticancer compounds to cancer cells residing in bone tissue or in tissue surrounding bone by administering to a mammal an effective amount of a first compound that is toxic to a cancer cell, conjugated to a second compound having a high affinity for bone tissue such that the first compound is delivered to, and exerts its toxic effect primarily in or near bone tissue. The invention is also directed to compounds that are effective for treating cancers when used in the methods of the present invention.

One embodiment of the invention are anticancer compounds derived from bradykinin receptor antagonists that have been conjugated with amino-bisphosphonate derivates to effectively localize the anticancer compounds to bone and surrounding soft tissue and methods of using these compounds to treat cancer in a mammal. These amino-bisphosphonate anticancer conjugates have a high affinity to bone and are therefore useful in the delivery of cytotoxic compounds to bone cancer cells including, but not limited to, bone metastases stemming from solid tumors such as prostate, breast, lung, renal, thyroid, osteosarcoma and skin cancer, as well as bone cancers resulting from liquid tumors such as myeloma, leukemia and lymphoma. The high affinity to bone shown by these anticancer compounds also makes them useful for the treatment of tumors formed in adjacent stromal cell compartments and the prevention of further growth, implantation or seeding of tumor cells in these areas. Additionally, because of their high affinity to bone and cancer cells, these anticancer amino-bisphosphonates may be modified as effective imaging agents to detect locations of tumor cells and their unique host microenvironment.

Another embodiment of the invention are bisphosphonate compounds derivatized to target and deliver antineoplastic compounds to bone and the surrounding soft tissue making it possible to treat and co-target bone cancers and their microenvironment, particularly prostate cancer bone metastases, be they hormone refractory or hormone-independent. Treatment with these bisphosphonate compounds may also be used in combination with other commonly-known hormone treatments, including, but not limited to, chemotherapy, radiation therapy, biological therapy and or surgery to improve pain management, survival and quality of life of prostate cancer patients. These anticancer-amino-bisphosphonate conjugates are particularly effective to kill or inhibit the growth of prostate cancer cells and prostate cancer metastases residing in bone and in the vicinity of bone.

### BRIEF DESCRIPTION OF THE DRAWINGS

FIG. 1. Survivin expression is associated with bone metastasis in human prostate cancer specimens and the experimental model. (A) IHC staining of survivin increased from well-differentiated to poorly differentiated prostate cancer primary tumors, and further increased in bone metastatic tumors. (B) Western blot analysis of survivin expression in human prostate cancer cell models. Survivin was increased in bone metastatic prostate cancer cells C4-2 and ARCaPM cells, compared to their parental LNCaP and ARCaPE cells, respectively. (C) IHC expression of survivin was increased in bone metastatic ARCaPM tumors compared to the primary tumors.

FIG. 2. BKM1740, an acyl-tyrosine bisphosphonate amide derivative, inhibits in vitro proliferation of C4-2 and ARCaPM cells. BKM1740 effects on proliferation of C4-2 and ARCaPM cells. The prostate cancer cells were cultured in the presence of BKM1740 at indicated concentrations for various durations. The effects of BKM1740 treatment on cell numbers were evaluated using MTS assay.

FIG. 3. BKM1740 induces apoptosis in metastatic Prostate cancer cells. (A) C4-2 cells were treated with BKM1740 at the indicated concentrations for 24 h, and Annexin V FACS analysis was performed. Right panel shows the percentage of apoptotic cells induced by BKM1740 treatment. (B) Western blot analysis of activation of caspase pathways. C4-2 cells were exposed to 5 μM BKM1740 for 12 h. Activated caspase-3, -8, and -9, and cleavage of PARP were detected by the increased banding of proteins at 17, 40, 35, and 89 Kda, respectively.

FIG. 4. BKM1740 inhibits survivin expression in metastatic prostate cancer cells. (A) BKM1740 specifically suppresses survivin expression at both RNA and protein levels. Left panel, C4-2 cells were exposed to 52404 BKM1740 for 12 h. Total RNA were collected and analyzed by RT-PCR for survivin, Mcl-1 and VEGF, with GAPDH as loading control. Right panel, C4-2 cells were treated with 5 μM BKM1740 for 24 h, and total lysates were analyzed for the expression of survivin and Mcl-1. β-actin was used as loading control. (B) C4-2 cells were co-transfected with pSurvivin-luc1430 and pRL-TK (internal control) for 48 h prior to exposure to BKM1740 at the indicated concentrations for a further 24 h incubation. Total lysates were analyzed for luciferase activity induced by the survivin promoter, and normalized to the Renilla luciferase activity.

FIG. 5. BKM1740 induces regression of Prostate cancer skeletal tumor in C4-2 mouse xenografts. (A) Intraperitoneal injection of BKM1740 reduced serum PSA in mice bearing C4-2 tumors in mouse skeleton, in comparison with control group after 8 weeks treatments (p<0.05). (B) Representative chromatograms of the bone-bearing tumors in each group as detected by x-ray, showing BKM1740 treatment improves the tumor x-ray appearances in comparison with control group.

FIG. 6. BKM1740 treatment exhibits growth inhibitory and pro-apoptotic activities against C4-2 tumor xenografts in mice. BKM1740 treatment inhibited cell proliferation (Ki67), induced apoptosis (M30), and suppression of survivin expression in vivo as analyzed by immunohistochemical analysis. Detailed comparative quantification of the BKM1740 treatment as opposed to controls on the expression of markers of cell proliferation, apoptosis and survivin expression are shown (p<0.05).

### DESCRIPTION OF THE INVENTION

One embodiment of the invention is a compound of Formula (I):

B-L-A (I)
or a pharmaceutically acceptable salt thereof,
   wherein,
B is a fragment of an anti-cancer bradykinin receptor antagonist having the chemical formula:

F5c-OC2Y-

F5c-OC2Y-Pipe-

F5c-OC2Y-Arg-

Bcpa-Bip-

Bipa-Bip-

F5c-Bip-

Pcin-Bip-

Pcn-Bip-

Pya-Bip-

Bcpa-OC2Y-

Bipa-OC2Y-

Pcn-OC2Y-

F5c-Bip-Pipe-

Pcn-Bip-Pipe-

Bcpa-Bip-Pipe-

Bipa-Bip-Pipe-

Pcin-Bip-Pipe-

F5c-ChG-Arg-

F5c-Bip-Arg-

Bcpa-Bip-Arg-

F5c-PFF-Arg-

Fmba-OC2Y- or,

F5c-D-OC2Y-

Wherein the abbreviations in these chemical formulae are:

Bcpa=bis(4-Chlorophenyl)acetyl

Bip=13-(4-Biphenylyl)alanine

Bipa=4-Biphenylacetyl

ChG=α-Cyclohexylglycine

D and L=amino acid configuration

F5c=2,3,4,5,6-Pentafluorocinnamoyl

Fmba=2-Fluoro-α-methyl-4-biphenylacetyl

OC2Y=O-2,6-Dichlorobenzyl-tyrosine

Pcin=4-Phenyl cinnamoyl

Pcn=α-Phenyl cinnamoyl

PFF=p-Fluorophenylalanine

Pipe=Piperidine

Pya=trans-3-(3-Pyridyl)acryloyl

TFA=Trifluoroacetic acid

L is a linking moiety having the chemical structure -piperidinyl-; and,

A is an aminobisphosphonate having the chemical structure:

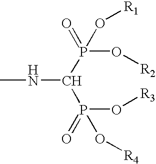

wherein R₁, R₂, R₃ and R₄ are independently H, methyl or ethyl.

In one embodiment of the invention, the linking moiety L is absent and Formula I may be expressed as B-A, wherein the anti-cancer bradykinin receptor antagonist fragments B, as listed above, are bound directly to the aminobisphosphonate moiety A, described above, without an intervening linking moiety.

A preferred embodiment of the present invention is a compound of Formula (I) or pro-drug forms thereof having the chemical structure:

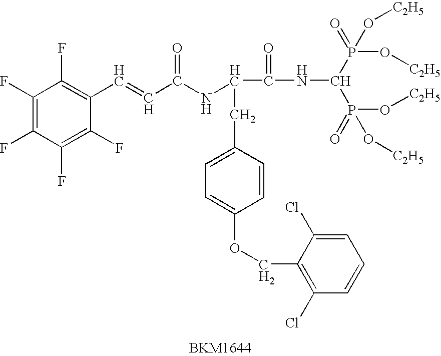

Another preferred embodiment of this invention is a compound of Formula (I), or pharmaceutically acceptable salts or pro-drug forms thereof having the chemical structure:

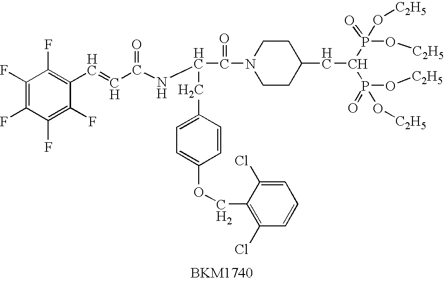

Particularly preferred embodiments of the invention include the compounds: [[[N-(2,3,4,5,6-pentafluorocinnamoyl)-O-[(2,6-dichlorophenyl)methyl]-L-tyrosyl]amino]methylene]bis(phosphonic acid) tetraethyl ester, 1-{[N-(2,3,4,5,6-pentafluorocinnamoyl)-O-(2,6-dichlorobenzyl)]-L-tyrosyl}-4-[bis(diethoxy-phosphono)]methylaminopiperidine, and pharmaceutically acceptable salts and pro-drug forms thereof.

Related embodiments of the present invention include methods of treating cancer in a mammal by contacting the mammal with at least one aminobisphosphonate compound of the present invention. The methods of the invention may also include contacting cancer cells that have been cultured in vitro or in vivo, such as in cell culture or in an animal, with a compound of the present invention to kill or inhibit the growth of the cancer cells.

In a preferred embodiment, the methods of the invention include inhibiting the growth of prostate cancer cells by contacting the prostate cancer cells, including metastases of prostate cancer, with one of the compounds of Formula I.

In another embodiment, the methods of the invention include activation of at least one of caspases 3, 9 and PARP, inhibition of the expression of survivin, and/or inducing apoptosis in a cell by contacting the cell with one of the compounds of Formula I.

The methods of the invention also include the administration of a therapeutically effective amount of at least one of the compounds of the invention to a mammal in need of such treatment. These methods of administration may include the administration of therapeutically effective amounts of pharmaceutically-acceptable salts, or solvates, or metabolites or prodrug forms of a compound of Formula Ito a mammal to treat a disease state in the mammal, such as a cancer.

The invention also encompasses pharmaceutical compositions containing at least one of the compounds of Formula I and a pharmaceutically-acceptable carrier or excipient and methods of killing or inhibiting the growth of a cancer cell by contacting the cell with at least one of these pharmaceutical compositions. The compounds present in these pharmaceutical compositions may be in the form of pharmaceutically-acceptable salts, or solvates, or metabolites or prodrug forms of a compound of Formula I described above.

The present invention provides methods of treating cancer in a mammal by administering a therapeutically effective amount of one of the compounds of Formula I to the mammal. These methods may include killing or inhibiting the growth of cancer cells in the mammal. While not intending to be constrained by theory, the mechanisms by which the compounds of Formula I inhibit or kill cancer cells is understood to include both the induction of apoptosis and the blockade of the expression of survivin, an anti-apoptotic protein that is often up-regulated in prostate cancer progression, in these cells. The induction of apoptosis is accompanied by the activation of caspases 3, 9 and PARP in these cells. These cellular effects result in reduced growth and inhibition of the malignant phenotype of the cell and ultimately, death of the cell. Prostate cancer cells having metastasized to bone are particularly susceptible to this mechanism of retarding cell growth or inducing cellular death by the accumulation of bradykinin antagonists in and around the bone of the mammal.

The methods of treating a mammal with a cancer through the administration of the compounds of Formula I may include the administration of an effective amount of an additional chemotherapeutic agent such as, but not limited to, acalacinomycin, alitretinoin, allopurinol, altretamine, anastrozole, arsenic trioxide, asparaginase, busulfan, calusterone, camptothecin, capecitabine, carmofur, cladribine, dacarbazine, dexrazoxane, docetaxel, doxifloridine, doxorubicin, dromostanolone, epirubicin, estramustine, etoposide, exemestane, floxuridine, fludarabine, fluorouracil, fulvestrant, gemcitabine, homoharringtonine, hydroxycamptothecin, hydroxyurea, irinotecan, letrozole, levamisole, mesna, mitotane, mitoxantrone, oxaliplatin, paclitaxel, pipobroman, pirarubicin, sarmustine, semustine, tamoxifen, tegafur-uracil, temozotomide, teniposide, testolactone, thioguanine, thiotepa, topotecan, valrubicin, vinblastine, vincristine, vindesine, and vinorelbine.

The methods of treating a mammal with a cancer through the administration of the compounds of Formula I may also include the application of radiation therapy, biological therapy, phototherapy and/or surgery.

The bradykinin antagonist-aminobisphonphonate conjugates of this invention may have one or more asymmetric centers or planes and it will be appreciated by those skilled in the art that compounds of the invention having a chiral center may exist in, and be isolated in, optically active and racemic forms. Additionally, some compounds may exhibit polymorphism. It is to be understood that the present invention encompasses any chiral (enantiomeric and diastereomeric) racemic, optically-active, polymorphic, or stereoisomeric forms, or mixtures thereof, of the compounds of the present invention. Many geometric isomers of olefins, C═N double bonds, and the like can also be present in these compounds, and all such stable isomers are also contemplated in the present invention. Methods of preparing optically active forms (for example, by resolution of the racemic form by recrystallization techniques, by synthesis from optically-active starting materials, by chiral synthesis, or by chromatographic separation using a chiral stationary phase) are well known in the art. Methods to determine anti-cancer and anti-tumor activity using the in vitro and in vivo tests described herein, or using other similar tests are also well known in the art. In each instance, all chiral, (enantiomeric and diastereomeric) and racemic forms and all geometric isomeric forms of a structure are intended, unless the specific stereochemistry or isomer form is specifically indicated in this disclosure.

In addition, the invention also includes solvates, metabolites, and pharmaceutically acceptable salts of compounds of Formula I.

“Pharmaceutically-acceptable salts” refer to derivatives of the disclosed compounds in which the parent compound is modified by making acid or base salts thereof. Examples of pharmaceutically-acceptable salts include, but are not limited to, mineral or organic acid salts of basic residues such as amines, or alkali or organic salts of acidic residues such as carboxylic acids. Pharmaceutically-acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically-acceptable salts are those forms of compounds, suitable for use in contact with the tissues of human beings and animals without causing excessive toxicity, irritation, allergic response, or other problems or complication, commensurate with a reasonable benefit/risk ratio.

Pharmaceutically-acceptable salt forms of compounds provided herein are synthesized from the parent compound which contains a basic or acidic moiety by conventional chemical methods. Generally, such salts are prepared, for example, by reacting the free acid or base forms of these compounds with a stoichiometric amount of the appropriate base or acid in water or in an organic solvent, or in a mixture of the two. Generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is incorporated herein by this reference. Of the particularly preferred embodiments of the invention, BKM 1644 is a neutral compound with no functional groups to form salts, while BKM 1740 is basic and forms salts with acids.

“Prodrugs” are considered to be any covalently bonded carriers, which release the active parent drug of Formula (I) in vivo when such prodrug is administered to a mammalian subject. Prodrugs of the compounds of Formula (I) are prepared by modifying functional groups present in the compounds in such a way that the modifications are cleaved, either in routine manipulation or in vivo, to the parent compounds. Prodrugs include compounds wherein hydroxy, amine, or sulfhydryl groups are bonded to any group that, when administered to a mammalian subject, cleaves to form a free hydroxyl, amino, or sulfhydryl group, respectively. Examples of prodrugs include, but are not limited to, acetate, formate and benzoate derivatives of alcohol and amine functional groups in the compounds of Formula (I), and the like. Compounds that function effectively as prodrugs of the compounds of Formula I may be identified using routine techniques known in the art. For examples of such prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8, 1-38 (1992); d) H. Bundgaard, et al., Journal of Pharmaceutical Sciences, 77:285 (1988); and e) N. Kakeya, et al., Chem. Pharm. Bull., 32: 692 (1984), each of which is specifically incorporated herein by reference.

The term “solvate” refers to an aggregate of a molecule with one or more solvent molecules. A “metabolite” is a pharmacologically active product produced through in vivo metabolism in the body of a specified compound or salt thereof. Such products may result for example from the oxidation, reduction, hydrolysis, amidation, deamidation, esterification, deesterification, enzymatic cleavage, and the like, of the administered compound. Accordingly, the invention includes metabolites of compounds of Formula I, including compounds produced by a process comprising contacting a compound of this invention with a mammal for a period of time sufficient to yield a metabolic product thereof.

The term “therapeutically effective amount” of a compound of this invention means an amount effective to prevent, treat, kill, reduce the growth or inhibit the malignant phenotype of neoplastic cells in a mammalian host.

The compounds of the present invention may be prepared in a number of ways well known to one skilled in the art of organic synthesis. The compounds of the present invention can be synthesized using the methods described below, together with synthetic methods known in the art of organic chemistry, or variations thereon as appreciated by those skilled in the art. The compounds of this invention may be prepared using the reactions and techniques described in Examples 1 and 2 of this disclosure. The reactions are performed in solvents appropriate to the reagents and materials employed and suitable for the transformation being effected. Also, in the description of the synthetic methods described below, it is to be understood that all proposed reaction conditions, including the choice of solvents, reaction temperature, duration of the experiments and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions on the use of substituents that are compatible with the reaction conditions will be readily apparent to one skilled in the art and alternate methods must then be used.

Also provided herein are pharmaceutical compositions comprising compounds of this invention and a pharmaceutically-acceptable carrier, which are media generally accepted in the art for the delivery of biologically active agents to animals, in particular, mammals. Pharmaceutically-acceptable carriers are formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted.

The compounds of the invention are effective in treating diseases over a wide dosage range and are generally administered in a therapeutically-effective amount. The dosage and manner of administration will be defined by the application of the compound and can be determined by routine methods of clinical testing to find the optimum dose. These doses are expected to be in the range of 0.001 mg/kg to 100 mg/kg of active compound. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

When employed as pharmaceuticals, the compounds of Formula I are administered in the form of pharmaceutical compositions and these pharmaceutical compositions represent further embodiments of the present invention. These compounds can be administered by a variety of routes including oral, rectal, transdermal, subcutaneous, intravenous, intramuscular, and intranasal. Preferably, the anti-cancer compounds of the present invention are administered via intratracheal instillation or aerosol inhalation when used to treat bone cancer. Such pharmaceutical compositions are prepared in a manner well known in the pharmaceutical art and comprise at least one active anti-cancer compound of Formula I.

The pharmaceutical compositions of the present invention contain, as the active ingredient, one or more of the compounds described by Formula I above, associated with pharmaceutically acceptable formulations. In making the compositions of this invention, the active ingredient is usually mixed with an excipient, diluted by an excipient or enclosed within a carrier, which can be in the form of a capsule, sachet, paper or other container. An excipient is usually an inert substance that forms a vehicle for a drug. When the excipient serves as a diluent, it can be a solid, semi-solid, or liquid material, which acts as a vehicle, carrier or medium for the active ingredient. Thus, the compositions can be in the form of solutions, syrups, aerosols (as a solid or in a liquid medium), ointments containing, for example, up to about 30% by weight of the active compound, soft and hard gelatin capsules, suppositories, sterile injectable solutions, and sterile packaged powders.

In preparing a formulation, it may be necessary to mill the anti-cancer compound to provide the appropriate particle size prior to combining with the other ingredients. If the anti-cancer compound is substantially insoluble, it ordinarily is milled to a particle size of less than 200 mesh. If the anti-cancer compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, gum Arabic, calcium phosphate, alginates, tragacanth, gelatin, calcium silicate, microcrystalline cellulose, polyvinylpyrrolidone, cellulose, sterile water, syrup, and methylcellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, about 0.1 mg to about 500 mg of the active ingredient of the present invention.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions that can be used include polymeric substances and waxes. The active ingredient can also be in microencapsulated form.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectal or vaginal cavity and release the active compound. Formulations of the present invention that are suitable for vaginal administration also include pessaries, tampons, creams, gels, pastes, foams or spray formulations containing such carriers as are known in the art to be appropriate.

Dosage forms for the topical or transdermal administration of compounds of this invention include powders, sprays, ointments, pastes, creams, lotions, gels, solutions, patches, and drops. The active ingredient may be mixed under sterile conditions with a pharmaceutically-acceptable carrier, and with any buffers, or propellants which may be required.

The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical formulations include those suitable for administration by inhalation or insufflation or for nasal or intraocular administration. For administration to the upper (nasal) or lower respiratory tract by inhalation, the compounds of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more compounds of the invention and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of the invention may be administered by means of nose drops or a liquid spray, such as by means of a plastic bottle atomizer or metered-dose inhaler. Typical of atomizers are the MISTOMETER™ (Wintrop) and MEDIHALER™ (Riker).

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

Pharmaceutical compositions of this invention suitable for parenteral administrations comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents that delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug is accomplished by dissolving or suspending the drug in an oil vehicle.

Injectable depot forms are made by forming microencapsulated matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

This invention further provides a method of treating a subject afflicted with a neoplasm or neoplastic disorder, by administering to the subject a pharmaceutical composition containing one or more of the compounds of Formula I described above. Such compositions generally comprise a therapeutically effective amount of a compound provided herein, that is, an amount effective to ameliorate, lessen, inhibit the growth of, or destroy, neoplastic tissue. Such amounts typically comprise from about 0.1 to about 1000 mg of the compound per kilogram of body weight of the subject to which the composition is administered. Therapeutically effective amounts can be administered according to any dosing regimen satisfactory to those of skill in the art.

Another aspect of the present invention provides methods of identifying effective and efficient treatments for prostate and/or bone cancer that function through the bradykinin receptor and associated signaling pathways, including the survivin gene. As explained above, antagonism of the bradykinin receptor is indicative of an effective cancer treatment and therefore, methods that indicate antagonism of this receptor or its downstream signaling pathway in response to a specific treatment may be indicative of treatments that suppress, inhibit or reduce the growth and survival of cancer cells. These methods include exposing test cells or organisms to potential or expected anti-cancer treatments and monitoring for effects, such as binding or antagonism, on the bradykinin receptor or related, downstream signaling pathway proteins. For example, following the treatment of tissue culture cells with an anticancer treatment, antagonism of the bradykinin receptor may be screened for in these cells. Similarly, these cells could be monitored for indications of c-src signaling blockade, and/or MAPK blockade mediated by intracellular calcium, GPCR, PLC, PI3K and/or PKC. Further, inhibition of survivin expression could also be monitored in these cells, as an indication of a treatment that inhibits or reduces cancer cell growth. This could be done by monitoring the expression of the survivin gene promotor through the use of an expression reporter that may be linked to the promotor in a single, heterologous nucleic acid construct. The introduction of a single vector containing the survivin promotor operably-linked to a reporter gene into the tissue culture cells, would be a typical means of monitoring the survivin gene expression following the treatment of these cells with a putative anti-cancer treatment, wherein inhibition of the survin promotor and survivin expression is indicative of an anti-cancer treatment. Specifically, because bradykinin receptor-mediated signaling involves the activation of intracellular transcriptional factors that recognize cis-elements appearing within the survivin promoter at +230 bp to +1430 bp, treatments that modulate the binding of these cis-elements to the survivin promotor may be indicative of anti-cancer therapies. Also, because transcription factors that bind to the survivin promoter in the region of +230 bp to +1430 bp can serve as survival factors for prostate and non-prostate cancer cells, these DNA binding proteins may also be targets for anti-cancer therapies, with the expectation that therapies that block or modulate the binding of these transcription factors will inhibit or arrest the growth of cancer cells.

The cancer therapies tested in these methods may include chemical or biological compounds, radiation therapies, phototherapies or combinations of these anti-cancer treatments.

Although the present invention has been described and exemplified in terms of certain preferred embodiments, other embodiments will be apparent to those skilled in the art. The invention is, therefore, not limited to the particular embodiments described and exemplified, but is capable of modification or variation without departing from the spirit of the invention. Additional objects, advantages, and novel features of this invention will become apparent to those skilled in the art upon examination of the following examples thereof, which are not intended to be limiting.

### EXAMPLES

The following chemical abbreviations are used throughout the following examples:

(AMDP(OEt)₄=tetraethyl aminomethylenediphosphonate
Boc=tert-butoxycarbonyl; (1,1-dimethylethoxy)carbonyl
BOP=benzotriazol-1-yloxy-tris(dimethylamino)phosphonium hexafluorophosphate
DCM=dichloromethane

### DIEA=N,N-diisopropylethylamine

### DMF=N,N-dimethylformamide

F5c=2,3,4,5,6-pentafluorocinnamoyl
HCl=hydrochloric acid
OC2Y═O-(2,6-dichlorobenzyl)-tyrosyl; 0-[(2,6-dichlorophenyl)methyl]-L-tyrosyl
Pipe=piperidine
Pipo=piperidin-4-one
TFA=trifluoroacetic acid

### Example 1

### Synthesis of [[[N-(2,3,4,5,6-pentafluorocinnamoyl)-O-[(2,6-dichlorophenyl)methyl]-L-tyrosyl]amino]methylene]bis(phosphonic acid) tetraethyl ester (BKM-1644, F5c-OC2Y-[AMDP(OEt)₄], C₃₄H₃₇Cl₂F₅N₂O₉P₂: 845.52)

To a solution of N-tent-butoxycarbonyl-O-(2,6-dichlorobenzyl)-L-tyrosine (Boc-OC2Y, 440.32 mg, 1.0 mmol), tetraethyl aminomethylenediphosphonate (AMDP(OEt)₄, 303.23 mg=255.03 μl, 1.0 mmol), and BOP (442.3 mg, 1.0 mmol) in acetonitrile (75 ml) was added N,N-diisopropylethylamine (360 μl, 2.0 mmol). The mixture was stirred at room temperature overnight then the solvent was removed in vacuum. The residue was partitioned between ethyl acetate (75 ml) and water (15 ml). The layers were separated and the organic phase was washed with 5% KHSO₄ (3×15 ml), brine (15 ml), 5% NaHCO₃ (3×15 ml), brine (15 ml). The organic layer was dried over anhydrous Na₂SO₄. The solvent was evaporated, affording a semi-solid product, (682 mg, 94.0%), [[[N-[(1,1-dimethylethoxy)carbonyl]-O-[(2,6-dichlorophenyl)methyl]-L-tyrosyl]amino]methylene]bis(phosphonic acid) tetraethyl ester (Boc-OC2Y-[AMDP(OEt)₄], C₃₀H₄₄Cl₂N₂O₁₀P₂: 725.54).

The N^{α}-Boc-group was cleaved according to the classical deprotection procedure.

The Boc-compound (682 mg, 0.94 mmol) was dissolved in 25% TFA in dichloromethane (DCM, 100 ml). After 7 minutes, the solution was concentrated under reduced pressure at room temperature and the residue was lyophilized from 12 ml of dioxane to give the crude product as a TFA salt ([[[O-[(2,6-dichlorophenyl)methyl]-L-tyrosyl]amino]methylene]bis(phosphonic acid tetraethyl ester), 650 mg, 93.5%, (OC2Y-[AMDP(OEt)₄].TFA, C₂₅H₃₆Cl₂N₂O₈P₂: 625.42+TFA=739.45). The crude product was purified by preparative HPLC on a C18 column to give the desired product (OC2Y-[AMDP(OEt)₄].TFA, as a white solid.

The OC2Y-[AMDP(OEt)₄].TFA was dissolved in 0.5 N cold HCl (10 ml) and the solution was filtered and lyophilized to obtain the OC2Y-[AMDP(OEt)₄].HCl salt (435 mg, 74.77%, C₂₅H₃₆Cl₂N₂O₈P₂: 625.42+HCl=661.88)

To a stirred mixture of OC2Y-[AMDP(OEt)₄].HCl (165.5 mg, 0.25 mmol), 2,3,4,5,6-pentafluorocinnamic acid (59.5 mg, 0.25 mmol) and BOP (110.5 mg, 0.25 mmol) in DMF (3 ml) was added DIEA (180 μl, 1.0 mmol). The mixture was stirred at room temperature overnight then the solvent was evaporated at reduced pressure. The residue was diluted with ethyl acetate (75 ml) and washed with 5% KHSO₄ (3×15 ml), brine (15 ml), 5% NaHCO₃ (3×15 ml) and brine (15 ml). After being dried over anhydrous Na₂SO₄, the organics were concentrated to dryness yielding the crude F5c-OC2Y-[AMDP(OEt)₄]. The crude product was purified by preparative HPLC on a C18 column to give the desired product after lyophilization from dioxane. (155 mg, 73.3%, as a white solid, F5c-OC2Y-[AMDP(OEt)₄], C₃₄H₃₇Cl₂F₅N₂O₉P₂: 845.52).

### Example 2

### Synthesis of 1-{[N-(2,3,4,5,6-pentafluorocinnamoyl)-O-(2,6-dichlorobenzyl)]L-tyrosyl}-4-[bis(diethoxyphosphono)]methylaminopiperidine (BKM-1740, F5c-OC2Y-Pipe[AMDP(OEt)₄], C₃₉H₄₆Cl₂F₅N₃O₉P₂: 928.65)

To a solution of N-tent-butoxycarbonyl-O-(2,6-dichlorobenzyl)-L-tyrosine (Boc-OC2Y, 880.64 mg, 2.0 mmol), 4-piperidone monohydrate hydrochloride (307.2 mg, 2.0 mmol) and BOP (884.6 mg, 2.0 mmol) in acetonitrile (75 ml) was added N,N-diisopropylethylamine (1.05 ml, 6.0 mmol). The mixture was stirred at room temperature overnight then the solvent was removed in vacuum. The residue was partitioned between ethyl acetate (75 ml) and water (25 ml). The layers were separated and the organic phase was washed with 5% KHSO₄ (3×25 ml), brine (25 ml), 5% NaHCO₃ (3×25 ml), brine (25 ml). The organic layer was dried over anhydrous Na₂SO₄. The crude, semi-solid product (Boc-OC2Y-Pipo, C₂₆H₃₀Cl₂N₂O₅: 521.44, 1030 mg, 98.8%) obtained after evaporation of the solvent in vacuum (t<40° C.), was submitted to reductive amination.

1-[N-tert-butoxycarbonyl-O-(2,6-dichlorobenzyl)-L-tyrosyl]-piperidin-4-one (1030 mg, 2.0 mmol) and tetraethyl aminomethylenediphosphonate (AMDP(OEt)₄, 606.4 mg=509.6 μl, 2.0 mmol) were dissolved in a mixture of methanol/acetic acid, 99:1 (35 ml) and sodium cyanoborohydride (377.1 mg, 6.0 mmol) was added portionwise during 45 minutes and the stirring was continued overnight. The solvent was evaporated at room temperature in vacuum and the residue was dissolved in ethyl acetate (75 ml). The solution was washed with saturated sodium bicarbonate solution (2×25 ml), water (25 ml), brine (25 ml) and dried on sodium sulfate. The solvent was evaporated, affording a yellow residue (1380 mg, 86.25%), 1-{[N-tert-butoxycarbonyl-O-(2,6-dichlorobenzyl)]-L-tyrosyl}-4-[bis(diethoxyphosphono)]methylaminopiperidine [(Boc-OC2Y-Pipe[AMDP(OEt)₄], C₃₅H₅₃Cl₂N₃O₁₀P₂: 808.67].

The N^{α}-Boc-group was cleaved according to the classical deprotection procedure. The Boc-compound (1.38 g, 1.7 mmol) was dissolved in 25% TFA in dichloromethane (DCM, 100 ml). After 10 minutes the solution was concentrated under reduced pressure at room temperature and the residue was lyophilized from dioxane (1.57 g). The crude product was dissolved in 3.2 mL of 80% acetonitrile, filtered using acrodisc syringe filter and purified by preparative HPLC column to give the desired product (OC2Y-Pipe[AMDP(OEt)₄].TFA, C₃₀H₄₅Cl₂N₃O₈P₂.TFA: 708.56+114.028=822.59; 790 mg, 56.4%) as a white solid. The following conditions were used: eluant A: H₂O/TFA (1000/1); eluant B: CH₃CN/TFA (1000/1); linear gradient time-program: eluant B from 30% to 46.7% within 10 min.; flow rate 10 mL/min; UV detection at 235 nm; injection 0.1 mL.

The OC2Y-Pipe[AMDP(OEt)₄].TFA salt was converted with 0.5 N HCl into the OC2Y-Pipe[AMDP(OEt)₄].HCl salt (C₃₀H₄₅Cl₂N₃O₈P₂: 625.42+HCl=745.02).

To a stirred mixture of OC2Y-Pipe[AMDP(OEt₄].HCl (111.8 mg, 0.15 mmol), 2,3,4,5,6-pentafluorocinnamic acid (35.72 mg, 0.15 mmol) and BOP (66.38 mg, 0.15 mmol) in DMF (2.5 ml) was added DIEA (105 μL, 0.6 mmol). The mixture was stirred at room temperature overnight and the solvent was evaporated at reduced pressure. The residue was diluted with ethyl acetate (75 ml) and washed with 5% KHSO₄ (3×15 ml), brine (15 ml), 5% NaHCO₃ (3×15 ml) and brine (15 ml). After being dried over anhydrous Na₂SO₄, the organics were concentrated to dryness yielding the crude F5c-OC2Y-Pipe[AMDP(OEt)₄] (126.8 mg, 91.0%, 85% purity by HPLC). The crude product was dissolved in 3.2 ml of 80% acetonitrile, filtered using acrodisc syringe filter and purified by preparative HPLC on a C18 column to give the desired product F5c-OC2Y-Pipe[AMDP(OEt)₄].TFA, C₃₉H₄₆Cl₂F₅N₃O₉P₂.TFA: 928.65+114.028=1042.68; 91.7 mg, 58.6%) as a white solid.

### Example 3

The studies described in this Example were designed to (a) determine the in vivo efficacy of BKM1740, a small-molecule, acyltyrosine bisphosphonate amide derivative, against human prostate cancer cell growth and survival in bone, and (b) investigate the molecular mechanism by which BKM1740 augments apoptosis in bone metastatic prostate cancer cells.

A. Survivin Expression Correlates with Bone Metastasis in Human Prostate Cancer Tumors.

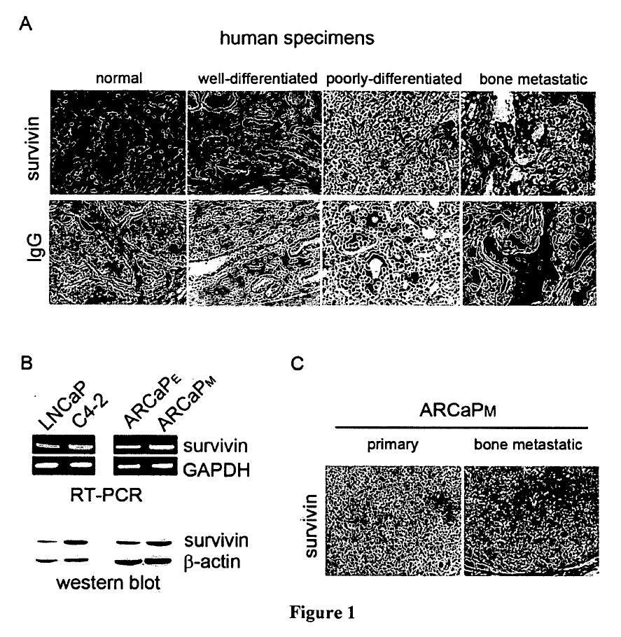

To investigate the clinicopathologic significance of survivin expression in human prostate cancer progression, immunohistochemistry protein expression of survivin was analyzed in primary and bone metastatic prostate cancer tissue. Well-differentiated prostate cancer was defined as Gleason score ˜6 and poorly-differentiated prostate cancer as Gleason score ˜8. In all specimens, survivin expression was undetectable or very low in normal/benign glands, but was increased in well-differentiated cancer to poorly-differentiated cancers. Importantly, survivin was highly expressed in all bone metastatic prostate cancer tumor specimens (FIG. 1A). These data indicate survivin expression is positively associated with prostate cancer progression, suggesting that survivin is a potential target for the treatment of prostate cancer bone metastasis.

Several lines of human prostate cancer cells were established that represent a continuum of prostate cancer progression closely mimicking the clinical pathophysiology of bone metastasis. Two lineage related sets of prostate cancer cells were used in this study: the LNCaP-C4-2 model, and the ARCaPEARCaPM model. RT-PCR and western blotting analyses indicated that survivin expression was elevated in highly bone metastatic prostate cancer cell lines C4-2 and ARCaPM, compared to the less invasive, parental cell lines LNCaP and ARCaPE, respectively (FIG. 1B). Further, ARCaPM cells were inoculated into athymic mice subcutaneously, which resulted in metastases to bone tissues within short latency. Survivin expression was examined by immunohistochemistry staining of the ARCaPM tumor specimens from either the primary site (subcutaneous injection) or metastatic bone. Consistently, survivin protein level was significantly increased in bone metastatic tumors over primary tumors (FIG. 1C). These data obtained from in vivo prostate cancer models validate a positive correlation between survivin expression and bone metastatic propensity observed in clinical situations.

### B. BKM1740 Induces Apoptosis in Metastatic Prostate Cancer Cells

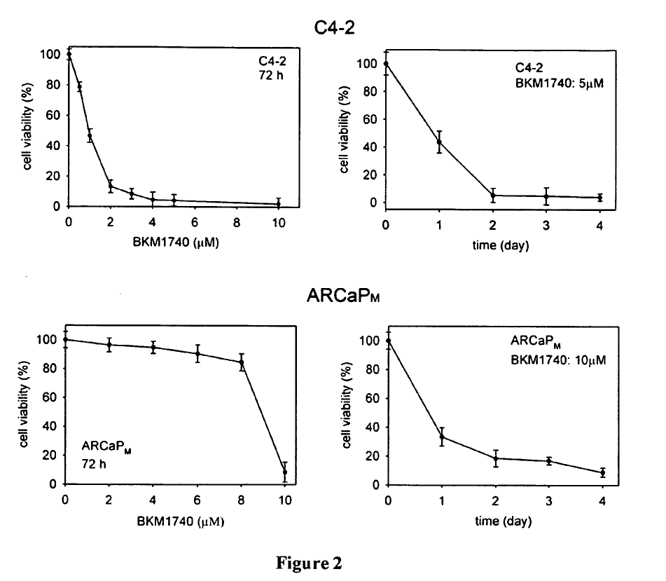

The cytotoxic effects of BKM1740 were first evaluated on bone metastatic prostate cancer cells. C4-2 and ARCaPM cells were exposed to the indicated concentrations of BKM1740 for various durations and cell proliferation was determined by MTS assay. BKM1740 was found to inhibit the in vitro growth of C4-2 and ARCaPM cells in a dose- and time-dependent manner, with 50% inhibition (IC50) observed at 2 μM and 9 μM, respectively (FIG. 2). Interestingly, compared with C4-2 cells, ARCaPM only responded to BKM 1740 treatment significantly within a narrow dose range (between 8 to 10 μM), suggesting this cell line is more resistant to the cytotoxicity of BKM1740.

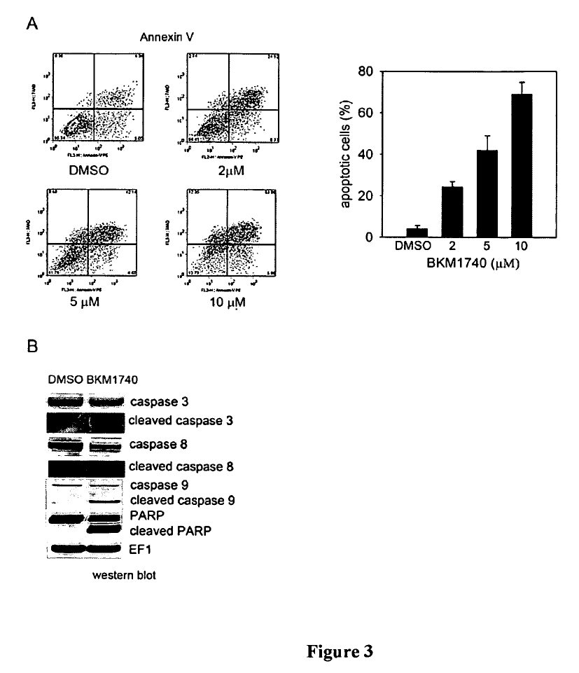

To further elucidate the mechanism for the effects of BKM1740 on prosate cancer cell viability, we determined annexin V expression, an indicator of apoptosis, in C4-2 cell treated with BKM1740 at the indicated concentrations for 24 h (FIG. 3A). FACS analysis indicated that BKM1740 treatment significantly induced apoptosis in C4-2 cells in a dose-dependent manner. Greater than 40% cell death can be achieved in 24 hours with 5 μM BKM1740 (FIG. 3A). Expression of caspases was determined by western blot analysis on C4-2 cell treated with 5 μM BKM1740. Activation of caspase-3, -8, and -9, as exhibited by increased cleaved protein bands at 17, 40, and 35 KDa, respectively, was observed after incubation with BKM1740 for 12 hours. Cleavage of PARP, an indicator of apoptosis and shown as a band at 89 Kd, was also increased significantly (FIG. 3B). These data suggest that BKM1740 induces apoptosis in metastatic PCa cell through a caspase-dependent pathway.

### C. BKM1740 Specifically Inhibits Expression of Survivin in Metastatic Prostate Cancer Cells

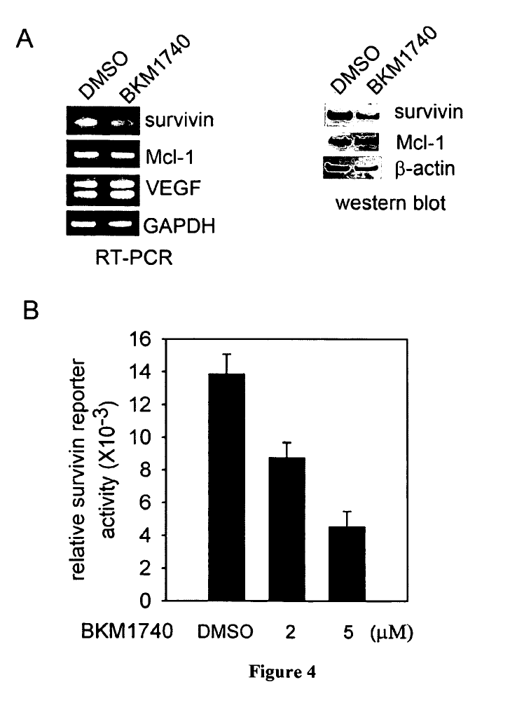

Multiple factors are involved in the regulation of cell death by apoptosis. To elucidate the specific signaling pathway(s) mediating the cytoxicity of BKM1740 in prostate cancer cells, expression of several anti-apoptotic proteins was analyzed in C4-2 cells treated with BKM1740 (FIG. 4A). RT-PCR assay indicated that BKM1740 significantly inhibited survivin expression at the mRNA level. BKM1740 treatment did not affect the expression of myeloid cell leukemia-1 (Mcl-1), an anti-apoptotic protein implicated in prostate cancer progression, or vascular endothelial growth factor (VEGF), a crucial angiogenic factor. Western blot analysis confirmed the inhibition of survivin protein expression following BKM1740 treatment in C4-2 cells (FIG. 4A).

C4-2 cells were transiently transfected with a survivin-luciferase reporter (pSurvivin-luc1430) containing a 1,430-bp region of human survivin promoter. The cells were further treated with BKM1740 at the indicated concentrations for 24 hours before the luciferase activity assay was performed. The data indicated that BKM1740 inhibited the survivin reporter activity in a dose-dependent manner (FIG. 4B), suggesting that survivin transcription was suppressed by BKM1740 treatment in C4-2 cells, which was consistent to the RT-PCR results (FIG. 4A). Taken together, these data demonstrate that BKM1740 specifically inhibits survivin expression in bone metastatic prostate cancer cells, which may mediate the activation of caspase-dependent apoptotic death caused by this compound.

### D. BKM1740 Treatment Inhibits In Vivo C4-2 Tumor Growth in Mouse Skeleton

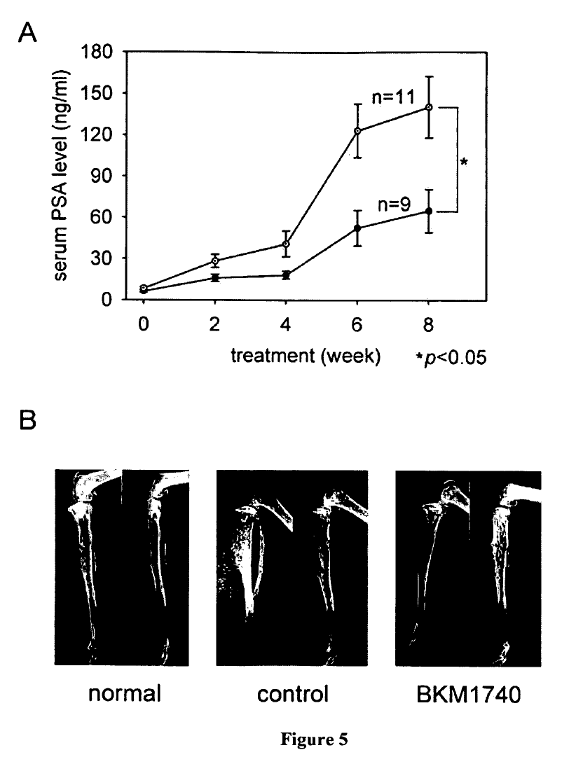

To evaluate the in vivo effect of BKM1740 against the growth of bone metastatic prostate cancer tumors, athymic nude mice bearing intratibia C4-2 xenografts were treated with BKM1740 at a dose of 5 mg/kg, three times per week, by the i.p. route. The treatment started on day 28 (4 weeks) after tumor inoculation, and continued for a duration of 8 weeks. Tumor growth and responsiveness to BKM1740 treatment were determined by serum PSA and x-ray of the skeleton. As shown in FIG. 5A, there was a significant reduction in serum PSA levels in the BKM1740-treated groups compared with vehicle control for 8 weeks (p<0.05). Representative examples of radiographs are shown in FIG. 5B. Compared to normal mouse bone, C4-2 tumor bearing bones treated with the vehicle control displayed a mixture of osteoblastic and osteolytic lesions. However, BKM1740-treated mouse bones had decreased osteolytic destruction and oestoblastic areas. The ratios of tumor areas were decreased, and the ratios of cortical bone and bone marrow were increased to the values found in normal bone. These x-ray results were consistent to the inhibitory effects of BKM1740 treatment on serum PSA levels in C4-2 tumor bearing mice. Notably, no obvious toxicities were induced by BKM1740 treatment, as reflected by the lack of body weight loss or infection in mice, suggesting a negligible in vivo acute toxicity of BKM1740.

### E. Immunohistochemistry Analysis of Human Prostate Cancer Xenografts Subjected to BKM1740 Treatment

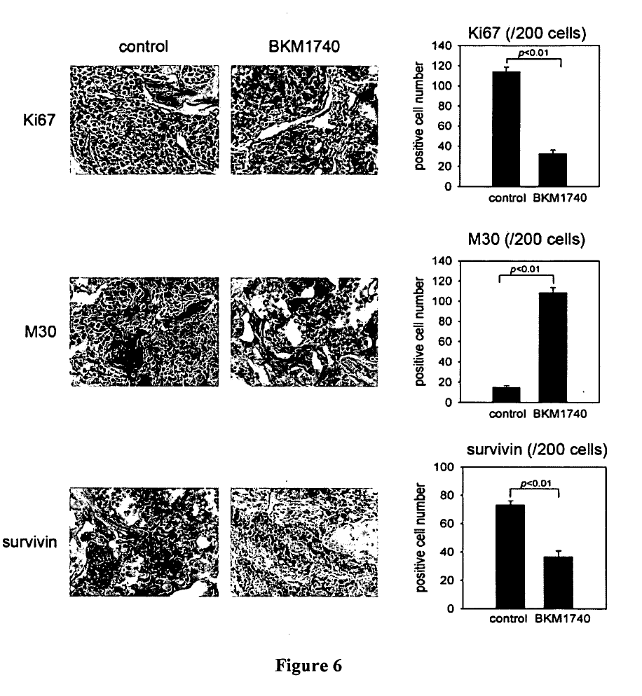

The effects of BKM1740 treatment on C4-2 tumor growth in tibia were confirmed by IHC analyses of the harvested tumor specimens at the termination of the experiments. IHC staining of mouse tibia indicated that compared with vehicle control, BKM1740 treatment markedly resulted in: 1) decreased cell proliferation (Ki67), and massive apoptosis (M30) in tumor tissues; and 2) significant inhibition of survivin expression. These differences are statistically significant (FIG. 6). These data confirm the in vivo effects of BKM1740 on C4-2 tumor growth are mediated by suppression of survivin expression and induction of apoptosis in prostate cancer tumors.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A compound having the chemical structure: B-L-A
or a pharmaceutically acceptable salt thereof,
wherein,
B is a fragment of an anti-cancer bradykinin receptor antagonist having a formula: F5c-OC2Y-, F5c-OC2Y-Pipe-, F5c-OC2Y-Arg-, Bcpa-Bip-, Bipa-, Bip-, F5c-Bip-, Pcin-Bip-, Pcn-Bip-, Pya-Bip-, Bcpa-OC2Y-, Bipa-OC2Y-, Pcn-OC2Y-, F5c-Bip-Pipe-, Pcn-Bip-Pipe-, Bcpa-Bip-Pipe-, Bipa-Bip-Pipe-, Pcin-Bip-Pipe-, F5c-ChG-Arg-, F5c-Bip-Arg-, Bcpa-Bip-Arg-, Bcpa-Bip-Arg, F5c-PFF-Arg-, Fmba-OC2Y-, or F5c-D-OC2Y-.
L is absent or is the linker piperidinyl; and,
A is an aminobisphosphonate having the chemical structure: 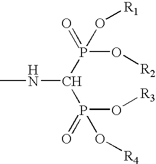
wherein R₁, R₂, R₃ and R₄ are independently H, methyl or ethyl.

2. The compound of claim 1 having the structure:

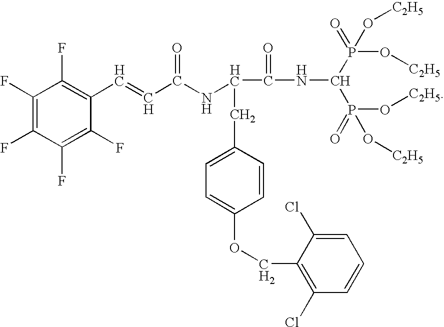

3. The compound of claim 1 having the structure:

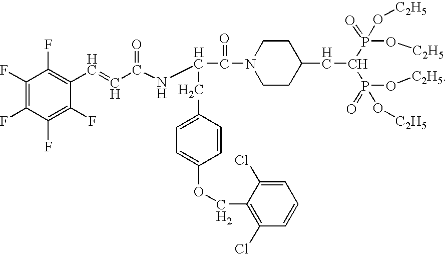

4. A pharmaceutical composition comprising a compound of claim 1 and at least one pharmaceutical excipient.

5. The method of claim 4, wherein the pharmaceutical composition further comprises an additional chemotherapeutic agent selected from the group consisting of acalacinomycin, alitretinoin, allopurinol, altretamine, anastrozole, arsenic trioxide, asparaginase, busulfan, calusterone, camptothecin, capecitabine, carmofur, cladribine, dacarbazine, dexrazoxane, docetaxel, doxifloridine, doxorubicin, dromostanolone, epirubicin, estramustine, etoposide, exemestane, floxuridine, fludarabine, fluorouracil, fulvestrant, gemcitabine, homoharringtonine, hydroxycamptothecin, hydroxyurea, irinotecan, letrozole, levamisole, mesna, mitotane, mitoxantrone, oxaliplatin, paclitaxel, pipobroman, pirarubicin, Sarmustine, semustine, tamoxifen, tegafur-uracil, temozotomide, teniposide, testolactone, thioguanine, thiotepa, topotecan, valrubicin, vinblastine, vincristine, vindesine, and vinorelbine.

6. A method of inhibiting the growth of cancer cells in a mammal comprising administering to the mammal a therapeutically effective amount of a compound of claim 1.

7. The method of claim 6, wherein the cancer cells are metastatic cancer cells.

8. The method of claim 6, wherein the cancer cells are bone cancer cells.

9. The method of claim 6, wherein the cancer cells are prostate cancer cells.

10. The method of claim 6, wherein the cancer cells are metastatic prostate cancer cells residing in bone.

11. The method of claim 6, comprising the additional step of administering to the mammal an effective amount of an additional chemotherapeutic agent selected from the group consisting of acalacinomycin, alitretinoin, allopurinol, altretamine, anastrozole, arsenic trioxide, asparaginase, busulfan, calusterone, camptothecin, capecitabine, carmofur, cladribine, dacarbazine, dexrazoxane, docetaxel, doxifloridine, doxorubicin, dromostanolone, epirubicin, estramustine, etoposide, exemestane, floxuridine, fludarabine, fluorouracil, fulvestrant, gemcitabine, homoharringtonine, hydroxycamptothecin, hydroxyurea, irinotecan, letrozole, levamisole, mesna, mitotane, mitoxantrone, oxaliplatin, paclitaxel, pipobroman, pirarubicin, Sarmustine, semustine, tamoxifen, tegafur-uracil, temozotomide, teniposide, testolactone, thioguanine, thiotepa, topotecan, valrubicin, vinblastine, vincristine, vindesine, and vinorelbine.

12. The method of claim 6, comprising the additional step of administering to the mammal an anti-cancer treatment selected from the group consisting of radiation therapy, phototherapy, biological therapy and surgical therapy.

13. A method of inhibiting the growth of cancer cells comprising contacting the cells with a compound of claim 1.

14. A method of activating at least one of caspases 3, 9 and PARP in a cell comprising contacting the cell with a compound of claim 1.

15. A method of inhibiting the expression of survivin in a cell comprising contacting the cell with a compound of claim 1.

16. A method of inducing apoptosis in a cell comprising contacting the cell with a compound of claim 1.

17. A method of identifying an inhibitor of cancer cell growth comprising:
a. exposing a cancer cell to an anti-cancer therapy; and,
b. monitoring an indicator of bradykinin receptor signaling selected from the group consisting of antagonism or blockade of bradykinin receptors, inhibition of c-src signaling, inhibition of MAPK, and decreased expression of a survivin gene,
wherein, inhibition of bradykinin receptor signaling is indicative of an effective anti-cancer therapy.

18. The method of claim 17, wherein the monitoring step comprises inhibition of the binding of transcriptional factors to a survivin gene promoter region.

19. The method of claim 18, wherein the transcription factor binding takes place in the region between +230 and +1430 of the survivin gene promoter.
